(19) United States
(12) Patent Application Publication
(10) Pub. No.: US 2005/0070762 A1
(43) Pub. Date: Mar. 31, 2005

(54) Electronic endoscope apparatus

(75) Inventors: Mitsuru Higuchi, Saitama-shi (JP); Minoru Iketani, Saitama-shi (JP)

Correspondence Address: SNIDER & ASSOCIATES, P. O. BOX 27613, WASHINGTON, DC, 20038-7613, US

(73) Assignee: Fuji Photo Optical Co., Ltd., Saitama-shi (JP)

(21) Appl. No.: 10/950,624

(22) Filed: Sep. 28, 2004

(30) Foreign Application Priority Data
Sep. 29, 2003 (JP) 2003-336719

(51) Int. Cl. A61B 1/04

(52) U.S. Cl. 600/109; 600/118

(57) ABSTRACT

In an electronic endoscope apparatus comprising a media drive unit for recording image data, which is given digital processing, on a recording medium, its aspect is that, while the free space of a recording medium is detected, a CCD pixel count (data volume of one image) of a connected electronic scope is judged, and hence, a current recordable amount on the recording medium is calculated by dividing the free space by the data volume of one image. Then, this recordable amount is displayed, for example on a part of four corners on the monitor. In addition, when a recording switch is pushed and the recording operation of one image is performed, the recordable amount is reduced by one, and this amount is displayed on the monitor. Owing to this, even when an electronic scope with a different pixel count is connected, it is possible to easily grasp and confirm the recordable amount.

### BACKGROUND OF THE INVENTION

The application claims the priority of Japanese Patent Applications No. 2003-336719 filed on Sep. 29, 2003 which is incorporated herein by reference.

1. Field of the Invention

The present invention relates to an electronic endoscope apparatus, and in particular, to the construction of an electronic endoscope apparatus where imaging signals obtained by solid state image pickup devices of electronic scopes with different pixel counts are outputted to a processor unit, and this processor unit can store digital image data on a recording medium.

2. Description of the Related Art

An electronic endoscope apparatus is equipped with a solid-state image pickup device such as a CCD (Charge Coupled Device) in an end section of an electronic scope (electronic endoscope). This CCD images an object on the basis of the illumination of light from light equipment. It becomes possible to display an image of the object on a monitor and to record a still image etc. on a recording apparatus, by outputting the imaging signal obtained by this CCD to a processor unit, and giving various kinds of picture processing in the processor unit.

As shown also in Japanese Patent Laid-Open No. 2000-287203, this kind of electronic endoscope apparatus performs not only analog processing for outputting an object image to a normal NTSC (PAL) system monitor, but also digital image processing to output the object image to various kinds of external digital equipment such as a personal computer monitor and use the image.

By the way, in an electronic endoscope apparatus, since an image (moving image and still image) was displayed on an NTSC system monitor, the signal processing was performed on the basis of the display pixel count (horizontal pixel count and vertical pixel count) of this NTSC system also in the above-mentioned digital image processing. But, in recent years, since CCDs which are solid state image pickup devices have been made to be in high pixel counts and high resolutions, it is proposed to form digital images where image information obtained by the CCDs with high pixel counts is employed efficiently. That is, in personal computers, for example, there are standards such as VGA (Video Graphics Array), XGA (Extended Graphics Array), and SXGA (Super XGA) whose display pixel counts are different. When forming an image signal corresponding to one of these standards, it becomes possible to use the image signal in external digital equipment. Then, in regard to the use in this kind of external equipment, it is proposed to record and store endoscope image data on recording media (information media) such as a PC card, Smart Media (registered trademark), a CompactFlash card (registered trademark), and an MO.

However, there is a problem that, since various kinds of pixel counts are used as the pixel counts of solid state image pickup devices mounted in electronic scopes, it is complicated to calculate and grasp a recordable amount during endoscopy even if the image data capacity of one image of an electronic scope connected and the recordable data capacity of a recording medium are known.

The present invention is made in view of the above-described problems, and aims at providing an electronic endoscope apparatus where an amount recordable on a recording medium can be grasped and confirmed easily even when an electronic scope having a different pixel count of a solid-state image pickup device is connected.

### SUMMARY OF THE INVENTION

In order to achieve the above-described object, the invention according to a first aspect is an electronic endoscope apparatus where an electronic scope in which various kinds of solid state image pickup devices with mutually different pixel counts are mounted is detachably connected to a processor unit, and object images are displayed on a monitor on the basis of output signals from the above-mentioned solid state image pickup devices, characterized in comprising a media drive unit for recording image data, which is given digital processing, on a recording medium, a control circuit which detects the free space of the recording medium mounted in this media drive unit, judges a pixel count of the solid state image pickup device in the above-mentioned electronic scope connected, and calculates a current amount of images recordable on the above-mentioned recording medium, and a display processing circuit for displaying the current amount recordable on the above-mentioned recording medium on a display unit on the basis of the control by this control circuit.

The invention according to a second aspect is characterized in that the control circuit subtracts a recordable amount whenever recording operation to the recording medium is performed, and that the display processing circuit displays this subtracted amount successively on the display unit.

According to the above-mentioned construction, while the free space (residual space) of a recording medium is detected, a pixel count (data volume of one image) of a connected electronic scope is judged. Hence, a current recordable amount is calculated by dividing the free space by the data volume of one image. Then, this recordable amount is displayed, for example, on a part of four corners on the monitor. In addition, when a recording switch is pushed and the recording operation of one image is performed, the recordable amount is reduced by one, and this amount is displayed on the monitor.

According to the electronic endoscope apparatus of the present invention, even when an electronic scope with a different pixel count of a solid state image pickup device is connected, it becomes possible to grasp and confirm easily the amount, recordable on a recording medium, on the display unit without calculating the amount from the data volume of one image of an electronic scope and the free space of the recording medium.

### BRIEF DESCRIPTION OF THE DRAWINGS

FIG. 1 is a circuit block diagram showing the structure of an electronic endoscope apparatus according to an embodiment of the present invention;

FIG. 2 is a schematic diagram showing the entire structure of the electronic endoscope apparatus according to the embodiment;

FIG. 3 is a diagram showing a display state of a recordable amount of object images and a recorded amount on a monitor of the embodiment;

FIG. 4 is a flowchart showing operation at the time of inserting a recording medium in a controller of the embodiment; and

FIG. 5 is a flowchart showing operation at the time of recording operation in the recording medium in the controller of the embodiment.

### DETAILED DESCRIPTION OF THE PREFERRED EMBODIMENTS

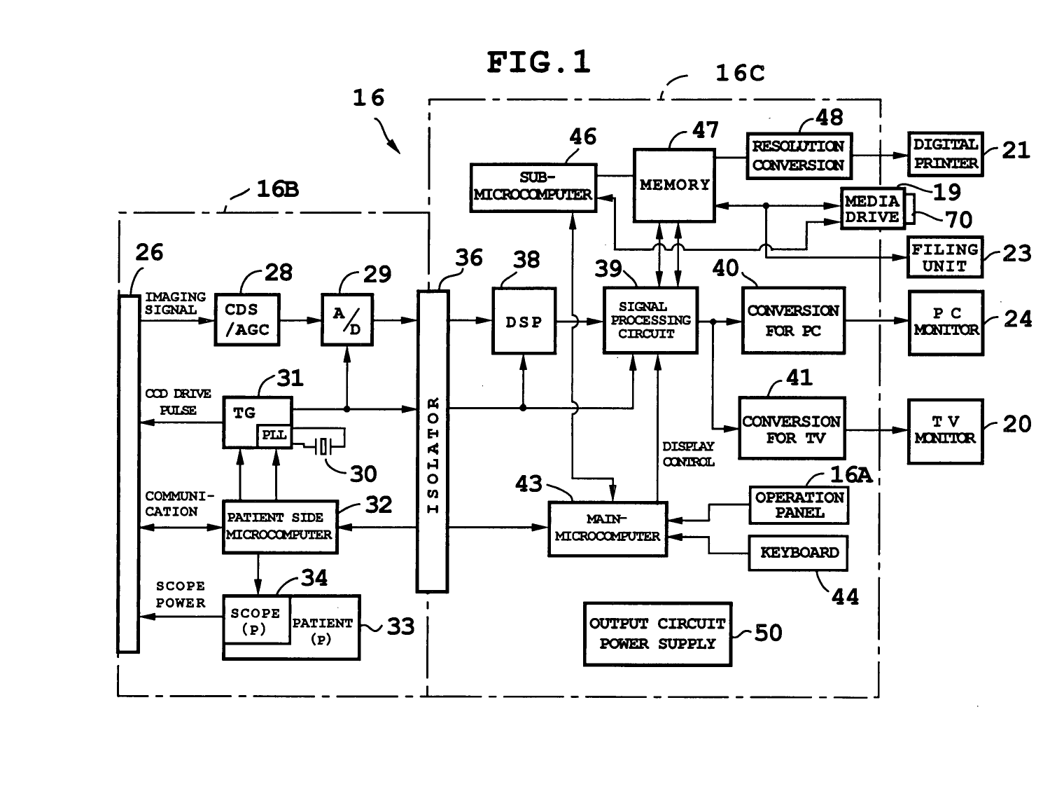

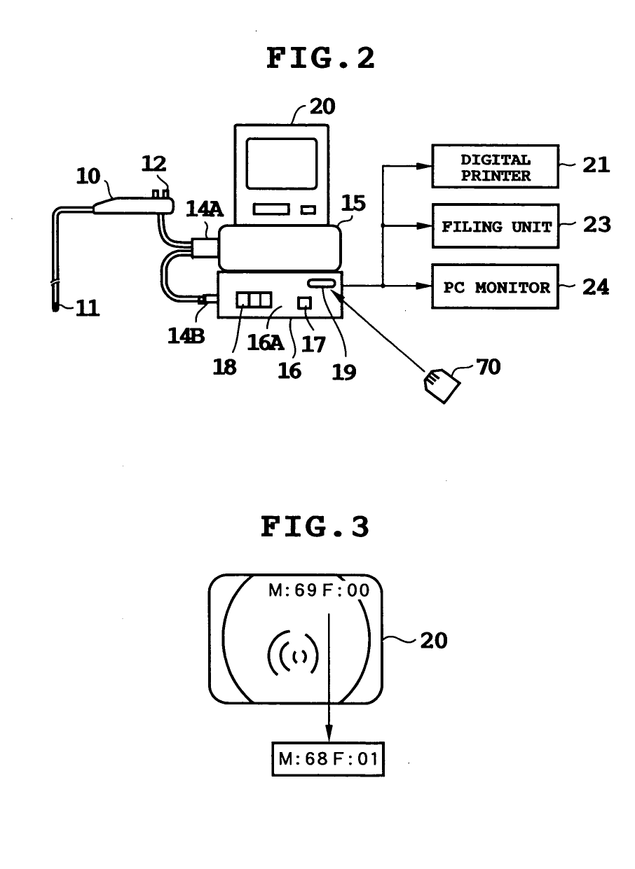

FIGS. 1 and 2 show the structure of an electronic endoscope apparatus according to an embodiment. First, on the basis of FIG. 2, the entire structure will be explained. As shown in FIG. 2, a CCD 11 which is a solid-state image pickup device is provided in an end section of an electronic scope (electronic endoscope) 10. As this CCD 11, various kinds of CCDs such as 350,000 pixels and 650,000 pixels are mounted. In an operation unit of this electronic scope 10, control switches such as a freeze/record button 12 are located. This electronic scope 10 is connected not only to light equipment 15 through a light guide connector 14A, but also to a processor unit 16 through a signal/power supply line connector 14B. The light of the above-mentioned light equipment 15 is supplied to the end section through a light guide located in the electronic scope 10. An object is imaged by the above-mentioned CCD 11 owing to illumination light emitted from this end section.

In a front operation panel (control panel) 16A of the processor unit 16, not only a main power supply switch (manual operation button) 17 and a scope power-off switch 18 are located, but also a loading slot of a media drive unit (drive) 19 which is located inside is provided. This media drive unit 19 writes and reads data between with a recording medium 70 which is called a PC card, a smart media, or the like. In addition, an NTSC system TV monitor 20, a digital printer 21, a filing unit 23, a personal computer (PC) monitor 24, and the like are connected to this processor unit 16.

FIG. 1 shows the detailed structure in the processor unit 16. This processor unit 16 has a patient circuit 16B which performs a predetermined picture processing, and an output circuit 16C which forms a signal correspondent to various kinds of output forms. The signal/power line connector 14B in the side of the above-mentioned electronic scope 10 is connected to the signal/power line connector 26. In the above-mentioned patient circuit 16B, when explaining from the upper part of the diagram, a CDS/AGC (correlation double sampling/automatic gain control) circuit 28 which samples and amplifies a picture signal inputted from the CCD 11, an A/D converter 29, a crystal oscillator 30, a timing generator (TG) 31 which forms a CCD drive pulse, supplied to the electronic scope 10, a synchronizing signal, etc., and a patient side microcomputer 32 which communicates with the electronic scope 10 and controls the patient circuit 16B are provided.

In this embodiment, since the example concerned is constructed so that only the power supply of the electronic scope 10 can be switched off, a patient power supply unit (P) 33 and a scope power supply unit 34 which supplies power to the electronic scope 10 through the signal/power line connector 26 (P) are provided. The switching of the power supply in this scope power supply unit 34 is controlled by the patient side microcomputer 32.

An output circuit 16C is connected to the patient circuit 16B through an isolator (electrical isolation means) 36. In this output circuit 16C, a DSP (Digital Signal Processor) 38 and a signal processing circuit 39 which give various kinds of image processing to the picture signal which is supplied from the A/D converter 29 and is digitized, a resolution conversion circuit 40 for PC which converts an output of this signal processing circuit 39 into predetermined resolution (for example, image sizes such as VGA and XGA) for displaying the output on the personal computer monitor 24, and a resolution conversion circuit 41 for TV which converts the output of this signal processing circuit 39 into an analog signal (Y/C signal etc.) in resolution (image size) for displaying the output on the NTSC system TV monitor 20 are provided. The signal processing circuit 39 has a character generator etc., generates characters (characters etc.) showing the amount of images already recorded (written) on the recording medium 70, the recordable image amount (remaining amount) in free space, and the like, and performs image mixing processing for displays the characters on a monitor screen.

In addition, a main microcomputer 43 which totally controls the circuits in the processor unit 16 and performs the display control of the recordable amount to the recording medium 70 is provided. To this main microcomputer 43, each control signal of the switches 17, 18, and the like which are located in the operation panel 16A is supplied. Then, a sub-microcomputer 46 which controls digital external equipment etc. (control of writing, reading, etc. of image data to the below-mentioned memory 47), and detects the free space of the recording medium 70, and image storage memory 47 which can store at least one set of examination data (for example, nearly 100 images) so as to output examination images are provided.

In order to receive an output of this image storage memory 47, the media drive unit 19 is connected. That is, the sub-microcomputer 46 accesses the recording medium 70 inserted in the recording medium drive unit 19 to detect the free space of this recording medium 70. On the other hand, so as to output the examination images to the digital printer 21 mentioned above, a resolution conversion circuit 48 which forms a digital image signal corresponding to standards such as VGA, XGA, and SXGA is provided in the image storage memory 47. In addition, an output circuit power supply unit (P) 50 is located in this output circuit 16C.

The embodiment has the above structure, and its operation will be described with referring to FIGS. 3 to 5. First, when the main power supply switch 17 of the operation panel 16A is pushed, power is supplied from the power supply units 50, 33, and 34 to each circuit, and image pickup by the CCD 11 at the end of the electronic scope 10 is started. A signal outputted from this CCD 11 is sampled by the CDS/AGC circuit 28, is converted into a digital signal by the A/D converter 29, and thereafter, is given various kinds of image processing by the DSP 38 and signal processing circuit 39. This picture signal is supplied through the resolution conversion circuit 40 for PC to the PC monitor 24, or through the resolution conversion circuit 41 for TV to the TV monitor 20, and the object image is displayed on each monitor. Then, when the freeze switch of the freeze/record button 12 of the electronic scope 10 which is a first step is pushed, the still images contained in the image memory in each of the resolution conversion circuits 40 and 41 (frame memory etc.) are displayed on the TV monitor 20 or PC monitor 24. In addition, this image data at the time of freeze is supplied also to the image storage memory 47.

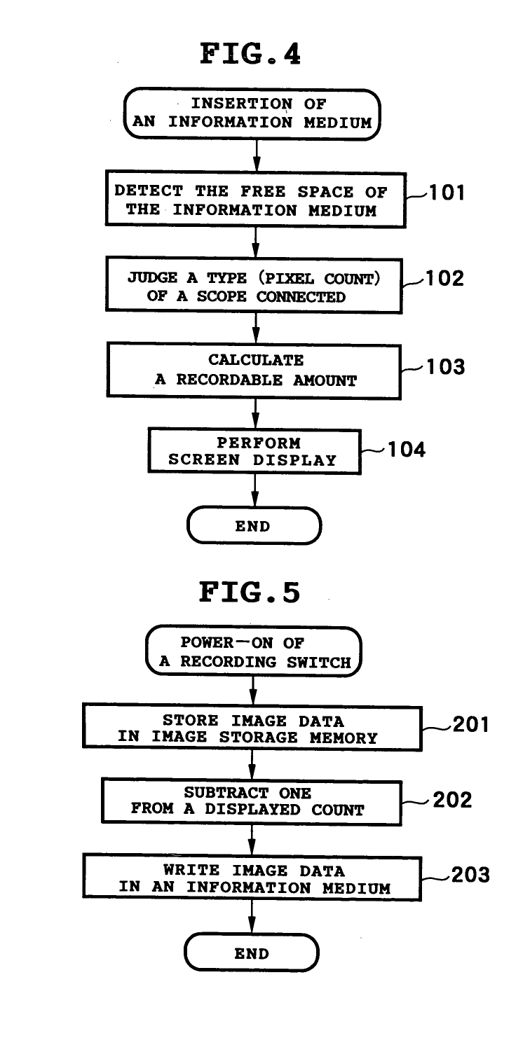

On the other hand, as shows in FIG. 4, when the recording medium 70 is inserted into the media drive unit 19 (or when the main power supply switch 17 is turned on), the sub-microcomputer 46 detects the free space in this recording medium 70 (step 101) to transmit this free space signal to the main microcomputer 43. This main microcomputer 43 communicates with the electronic scope 10 through the patient side microcomputer 32 to judge the pixel count of the CCD 11 from the identification data of this electronic scope 10 (step 102). Then, the main microcomputer 43 calculates a recordable amount on the basis of a data compression rate beforehand set with the operation panel 16A etc. (step 103).

For example, when the CCD 11 is 650,000 pixels (corresponding to the SXGA standard), one image size is 1280 (horizontal pixels)×960 (vertical pixels)×3 (corresponding to three chrominance signals of R, G, and B)=3.6864 MB (byte) in case of non-compression. Hence, assuming that the free space of the recording medium 70 is 256 MB, a recordable amount is 256/3.6864 69.4, which becomes 69 images. For example, when the CCD 11 is 350,000 pixels (corresponding to the VGA standard), one image size is 640 (horizontal pixels)×480 (vertical pixels)×3=0.9216 MB (byte). Hence, assuming that the free space of the recording medium 70 is 256 MB, a recordable amount is 256/0.9216 277.7, which becomes 277 images.

Then, at step 104, the screen display processing of this recordable amount and the amount already recorded is performed. In this embodiment, characters which show each amount are generated by the character generator in the signal processing circuit 39 and are mixed in a current image. Then, as shown in FIG. 3, the amount M recordable in the free space of the recording medium 70 and the amount F already recorded are displayed as “M: 69 (or M: 277)” and “F: 00” respectively on the screen of the monitor 20.

In addition, when the recording switch of the freeze/record button 12 which is a second step is pushed with looking at the display image of the TV monitor 20, as shown in FIG. 5, the still image currently supplied and written in the image storage memory 47 is maintained and stored (step 201). Then, the main microcomputer 43 subtracts one from the displayed amount (step 202). After that, when the still picture data stored in the image storage memory 47 is read by the control of the sub-microcomputer 46, the media drive unit 19 writes new still picture data in the recording medium 70 (step 203). Concurrently, the signal processing circuit 39 performs the display processing of the new amount by the control of the main microcomputer 43, and, as shown in FIG. 3, the recordable amount M (=68) (in the case of 650,000 pixels) and the amount F (=01) recorded now are displayed.

In addition, in the above-mentioned recording operation, the image storage memory 47 has the capacity of being able to store at least one examination set of image data. Hence, there arises the situation that, even after the endoscopy for a patient is ended, the recording operation to the recording medium 70 or external equipment 21 or 23 is not completed. Then, in this embodiment, only the power of the electronic scope 10 can be turned off. That is, when the scope power-off switch 18 of the operation panel 16A is pushed, the main microcomputer 43 outputs a command of scope power-off to the patient side microcomputer 32. Then, only the scope power supply unit 34 is turned off by the control of this patient side microcomputer 32.

At this time, since the patient power supply unit 33 and output circuit power supply unit 50 are not turned off, the outputting (writing) of the image data to the recording medium 70 or external equipment 21 to 23 is performed continuously. Accordingly, since the electronic scope 10 can be removed from the processor unit 16 before the recording operation is completed, it becomes possible to quickly perform the subsequent operations such as washing of the electronic scope 10, and connection of another electronic scope for the next examination.

Although the case that still images are recorded on the recording medium 70 is explained in the above-mentioned embodiment, the present invention can be applied also to the case that moving images are recorded. In addition, it is also possible to use a hard disk card, a MICRODRIVE (registered trademark), etc. as this recording medium 70. Furthermore, although the case that the media drive unit 19 is incorporated in the processor unit 16 is explained, it is possible to apply the present invention also when external media equipment having a media drive unit is connected to the processor unit 16. In addition, although a recordable amount and a recorded amount are displayed on the monitor 20, a liquid crystal display or the like may be also provided, for example, in the operation panel 16A as this display unit.

1. An electronic endoscope apparatus, comprising:
an electronic scope in which various kinds of solid state image pickup devices with different pixel counts are mounted;
a processor unit detachably connecting this electronic scope;
a monitor displaying object images imaged by the solid-state image pickup devices;
a media drive unit for recording image data, which is given digital processing, on a recording medium;
a control circuit which detects free space of the recording medium mounted in this media drive unit, judges a pixel count of the solid state image pickup device in the electronic scope connected, and calculates a current amount of images recordable on the recording medium; and
a display processing circuit for displaying the current amount recordable on the recording medium on a display unit on the basis of control by this control circuit.

2. The electronic endoscope apparatus according to claim 1, wherein the control circuit subtracts a recordable amount whenever recording operation to the recording medium is performed, and the display processing circuit displays this subtracted amount successively on the display unit.

3. The electronic endoscope apparatus according to claim 1, wherein the display processing circuit displays an amount already recorded on the recording medium.
